(19) United States
(12) Patent Application Publication
(10) Pub. No.: US 2005/0171429 A1
(43) Pub. Date: Aug. 4, 2005

(54) Method and system for very high frame rates in ultrasound B-Mode imaging

(76) Inventors: Prakash Parayil Mathew, Mukwonago, WI (US); Michael Charles Macdonald, New Berlin, WI (US)

Correspondence Address: MCANDREWS HELD & MALLOY, LTD, 500 WEST MADISON STREET, SUITE 3400, CHICAGO, IL, 60661, US

(21) Appl. No.: 10/758,848

(22) Filed: Jan. 16, 2004

(51) Int. Cl. A61B 8/00; A61B 8/12; A61B 8/14

(52) U.S. Cl. 600/437

(57) ABSTRACT

Certain embodiments include a system and method for an improved image acquisition rate in an ultrasound imaging system. The system includes an encoder for encoding an ultrasound signal with a code for transmission to form an encoded ultrasound signal, a transmitter for transmitting the encoded ultrasound signal, and a receiver for receiving an encoded echo signal produced based on the encoded ultrasound signal, wherein the receiver decodes the encoded echo signal to produce a decoded echo signal. The transmitter may include a transducer array for transmitting the encoded ultrasound signal. The receiver may include a separate transducer element or a dedicated transducer element of the transmitter transducer array. The system further includes a processor for determining a position of a scatterer based on a time of transmission of the encoded ultrasound signal, a time of reception of the encoded echo signal, and a strength of the encoded echo signal.

### BACKGROUND OF THE INVENTION

The present invention generally relates to ultrasound imaging. In particular, the present invention relates to attaining very high frame rates in ultrasound imaging.

Ultrasound is sound having a frequency that is higher than a normal person may hear. Ultrasound imaging utilizes ultrasound waves or vibrations in the frequency spectrum above normal human hearing, such as the 2.5-10 MHz range. Ultrasound imaging systems transmit ultrasound into a subject, such as a patient, in short bursts. Echoes are reflected back to the system from the subject. Diagnostic images may be produced from the echoes. Ultrasound imaging techniques are similar to those used in sonar and radar.

A medical ultrasound system forms an image by sequentially acquiring echo signals from ultrasound beams transmitted to an object being imaged. An individual beam is formed by transmitting a focused pulse and receiving the echoes over a continuous range of depths. An amplitude of an echo signal decreases significantly for signal reflectors located deeper in the object due to increased signal attenuation of intervening structures, such as intervening tissue layers. Therefore, a signal-to-noise ratio decreases since noise generated by the ultrasound system's signal amplifiers, for example, may not be reduced to arbitrary low levels.

Forming the best possible image at all times for different anatomies and patient types is important to diagnostic imaging systems. Poor image quality may prevent reliable analysis of the image. For example, a decrease in image contrast quality may yield an unreliable image that is not usable clinically. Additionally, the advent of real-time imaging systems has increased the importance of generating clear, high quality images.

B-Mode or “Brightness” mode is a common display format for an ultrasound image. Currently, B-Mode ultrasound imaging system transducers fire a narrow ultrasound beam or vector in a single direction. The transducer array then waits to listen to all echoes returning from reflectors along that same straight line. Strength of the return echoes is used to represent a reflectivity of an object. Reflectivity of an object, such as an anatomy of a patient, is typically calculated using a range equation. The range equation determines that time equals signal round trip divided by speed of sound in a subject medium. Current ultrasound systems utilize receive beamforming to reconstruct an object being imaged. That is, an ultrasound system listens for echo signals using a receiver which is then used for beam reconstruction and beam directional determination. The scheme of firing an ultrasound beam and listening for reflected echoes is repeated sequentially in a number of directions to span a two-dimensional section in an object space, such as an anatomical space. The ultrasound system paints each line that is determined from the reflected echo signals with a brightness level corresponding to a return echo signal strength. A complete set of vectors that sweep out a section of space constitutes a B-Mode frame. The reconstruction process is repeated to successively paint frames and achieve a standard real-time B-Mode display.

Current beam reconstruction operation is fundamentally limited by physics of sound wave propagation speed. For an ultrasound pulse fired (duration 1-10 μs, for example), the ultrasound system “waits” to “listen” for all echoes up to a certain depth in the object being imaged (for example, generally 100 mm). During the waiting period, a transducer that fired the ultrasound pulse is essentially performing receive beamforming on returned echoes.

Assuming a speed of sound in a tissue medium, for example, of 1.54 mm/μs, the system is “listening” for (200/1.54)μs˜=130 μs. Assuming that each vector takes 130 μs to acquire and there are 128 vectors in a two-dimensional frame, a frame takes about (130*128) μs or approximately 17 ms to acquire. Thus, a maximum frame rate in a typically system is about 60 frames per second (fps). There is a need for a method and system that improve image data acquisition frame rate.

Trade-offs may be made in current systems to improve frame rate. For example, lower penetration depth into an object being scanned, less lateral vector line density, a smaller region of interest, etc., may be used to achieve a higher frame rate. However, current methods are fundamentally limited by the “fire and listen” operation. Thus, a system and method that do not rely on the fire and listen method would be highly desirable.

Multi-line acquisition may be used to slightly improve data acquisition by acquiring data for multiple echo vectors from a single ultrasound beam firing. Higher frame rates may be achieved at the expense of lateral resolution and other trade-offs. Thus, there is a need for a system and method that provide increased frame rate while reducing other imaging trade-offs.

### BRIEF SUMMARY OF THE INVENTION

Certain embodiments of the present invention provide a method and system for an improved image acquisition rate in an ultrasound system. The method includes encoding an ultrasound signal with a code to produce an encoded ultrasound vector and transmitting from a first location the encoded ultrasound vector at a desired angle. The method also includes receiving at a second location an encoded echo signal produced in response to the encoded ultrasound vector and decoding the encoded echo signal using the code used to produce the encoded ultrasound vector.

The method may further include determining a position of a structure producing the encoded echo signal in response to an impact by the encoded ultrasound vector. The position of the structure may be determined based on a time of transmission of the encoded ultrasound vector, a time of reception of the encoded echo signal, and a strength of the encoded echo signal. An angle of transmission of the encoded ultrasound vector may also be used to determine the position of the structure. The time of transmission may be determined based on the code used to encode the appropriate ultrasound vector. In an embodiment, the method further includes transmitting from the first location a plurality of encoded ultrasound vectors at a plurality of angles, receiving at the second location a plurality of encoded echo signals produced in response to the plurality of encoded ultrasound vectors, and obtaining an image of an object based on the encoded ultrasound vectors and the encoded echo signals.

Another embodiment of the method includes encoding a plurality of ultrasound signals for a frame with distinct codes and sequentially transmitting from a first location the plurality of ultrasound signals at a plurality of angles. The method then includes receiving at a second location distinct from the first location a plurality of echo signals formed based the plurality of ultrasound signals and decoding the plurality of echo signals using the plurality of distinct codes. Each echo signal may be decoded using a code used to encode an ultrasound signal producing the echo signal. The plurality of echo signals may be decoded offline concurrently with transmission of ultrasound signals and reception of echo signals.

In an embodiment, the ultrasound imaging system includes an encoder for encoding an ultrasound signal with a code for transmission to form an encoded ultrasound signal, a transmitter for transmitting the encoded ultrasound signal, and a receiver for receiving an encoded echo signal produced based on the encoded ultrasound signal, wherein the receiver decodes the encoded echo signal to produce a decoded echo signal. The transmitter may include a transducer array, for example, for transmitting the encoded ultrasound signal. The receiver may include a separate transducer element or a transducer element of the transmitter transducer array, for example. If the receiver includes an element of the transmitter transducer array, that element is not used for transmitting the encoded ultrasound signal.

In an embodiment, the encoder encodes a plurality of ultrasound signals for transmission with a plurality of codes. The codes may include distinct codes for each ultrasound signal within a frame. The transmitter may essentially sequentially transmit a plurality of encoded ultrasound signals at an object being imaged. The transmitter may sequentially transmit the plurality of encoded ultrasound signals at a plurality of angles.

In an embodiment, the system further includes a processor for determining a position of a scatterer producing the encoded echo signal in response to an impact by the encoded ultrasound signal. The processor may determine the position of the scatterer based on a time of transmission of the encoded ultrasound signal, a time of reception of the encoded echo signal, and a strength of the encoded echo signal. The time of transmission may be determined based on the code used to encode the encoded ultrasound signal. The processor may also determine the position of the scatterer using an angle of transmission of the encoded ultrasound signal. The processor may determine position as additional encoded ultrasound signals are transmitted by the transmitter and additional encoded echo signals are received by the receiver.

### BRIEF DESCRIPTION OF SEVERAL VIEWS OF THE DRAWINGS

FIG. 1 illustrates a block diagram of an ultrasound imaging system used in accordance with an embodiment of the present invention.

FIG. 2 illustrates a method for ultrasound imaging in accordance with an embodiment of the present invention.

FIG. 3 illustrates an improved ultrasound transducer module for use in an ultrasound imaging system in accordance with an embodiment of the present invention.

FIG. 4 illustrates a flow diagram for a method for obtaining a very high frame rate in an ultrasound imaging system using separate transmitter and receiver elements in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

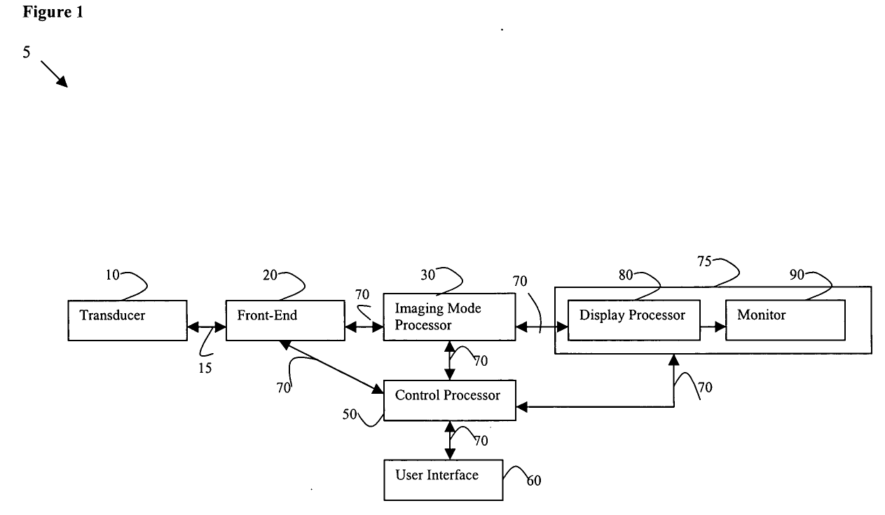

FIG. 1 illustrates a block diagram of an ultrasound imaging system 5 used in accordance with an embodiment of the present invention. The system 5 includes a transducer 10, a front-end subsystem 20, an imaging mode processor 30, a user interface 60, a control processor 50, and a display 75. The imaging mode processor 30 and the control processor 50 may be part of a back-end system. The transducer 10 is used to transmit ultrasound waves into a subject by converting electrical analog signals to ultrasonic energy. The transducer 10 may also be used to receive ultrasound waves that are backscattered from the subject by converting ultrasonic energy to analog electrical signals. The front-end subsystem 20 including a receiver, a transmitter, and a beamformer, is used to create transmitted waveforms, beam patterns, receiver filtering techniques, and demodulation schemes that are used for various imaging modes. The front-end 20 converts digital data to analog data and vice versa. The front-end 20 interfaces with the transducer 10 via an analog interface 15. The front-end 20 interfaces with the imaging mode processor 30 and the control processor 50 via a digital bus 70. The digital bus 70 may include several digital sub-buses. The digital sub-buses may have separate configurations and provide digital data interfaces to various parts of the ultrasound imaging system 5.

The imaging mode processor 30 provides amplitude detection and data compression for an imaging mode, such as B-mode imaging, M-mode imaging, BM-mode imaging, harmonic imaging, Doppler imaging, color flow imaging, and/or any other ultrasound imaging mode. The imaging mode processor 30 receives digital signal data from the front-end 20. The imaging mode processor 30 processes the received digital signal data to produce estimated parameter values. The estimated parameter values may be produced using the received digital signal data. The digital signal data may be analyzed in frequency bands centered at the fundamental, harmonics, or sub-harmonics of the transmitted signals to produce the estimated parameter values. The imaging mode processor 30 passes the estimated parameter values to a control processor 50 over the digital bus 70. The imaging mode processor 30 may also pass the estimated parameter values to the display 75 via the digital bus 70.

The display 75 includes a display processor 80 and a monitor 90. The display processor 80 accepts digital parameter values from the imaging mode processor 30 and the control processor 50. The display processor 80 may perform scan-conversion functions, color mapping functions, and tissue/flow arbitration functions, for example. The display processor 80 processes, maps, and formats the digital data for display, converts the digital display data to analog display signals, and passes the analog display signals to the monitor 90. The monitor 90 accepts the analog display signals from the display processor 80 and displays the resultant image. An operator may view the image on the monitor 90.

The user interface 60 allows user commands to be input by the operator to the ultrasound imaging system 5 through the control processor 50. The user interface 60 may include a keyboard, mouse, switches, knobs, buttons, track ball, and/or on screen menus, for example.

The control processor 50 is the central processor of the ultrasound imaging system 5. The control processor 50 interfaces to other components of the ultrasound imaging system 5 using the digital bus 70. The control processor 50 executes various data algorithms and functions for various imaging and diagnostic modes. Digital data and commands may be transmitted and received between the control processor 50 and other components of the ultrasound imaging system 5. In an alternative embodiment, functions performed by the control processor 50 may be performed by multiple processors and/or may be integrated into the imaging mode processor 30 and/or the display processor 80. In another embodiment, the functions of the processors 30, 50, and 80 may be integrated into a single personal computer (PC) backend.

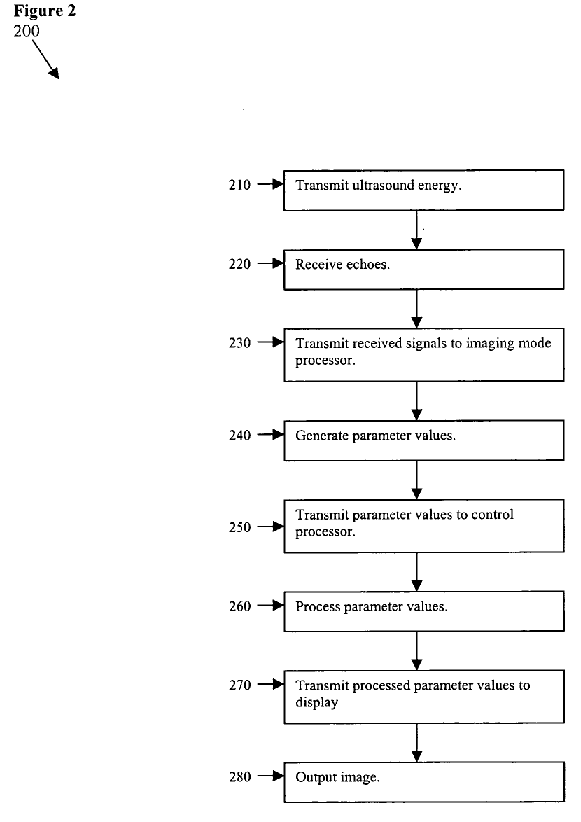

FIG. 2 illustrates a method 200 for ultrasound imaging in accordance with an embodiment of the present invention. First, at step 210, the transducer 10 transmits ultrasound energy into a subject, such as a patient. Then, at step 220, ultrasound energy or echoes backscattered from the subject are received. Signals are received at the front-end 20 in response to ultrasound waves backscattered from the subject. Transmission of ultrasound beams and reception of backscattered echo signals is discussed further below.

Next, at step 230, the received signals are transmitted from the front-end 20 to the imaging mode processor 30 using the digital bus 70. At step 240, the imaging mode processor 30 generates parameter values based on the received signals. Then, at step 250, the parameter values are sent to the control processor 50.

At step 260, the control processor 50 processes the parameter values for use in display, storage, and diagnostics at the display 75. The control processor 50 processes the image data parameter values to reduce artifacts and process resulting image(s). The control processor 50 and/or imaging mode processor 30 may compound image data to produce a compound image. For example, image data from a plurality of angles may be combined or averaged to produce a spatially compound image.

Next, at step 270, processed parameter values are transmitted to the display 75. The display processor 80 may also process parameter values from a plurality of focal zone images to produce a combined image in conjunction with and/or in addition to the control processor 50.

Finally, at step 280, a diagnostic image is produced and output at the monitor 90. The image may be stored, displayed, printed, and/or further transmitted, for example. The display processor 80 may produce the diagnostic image using the processed parameter values from the digital signal data.

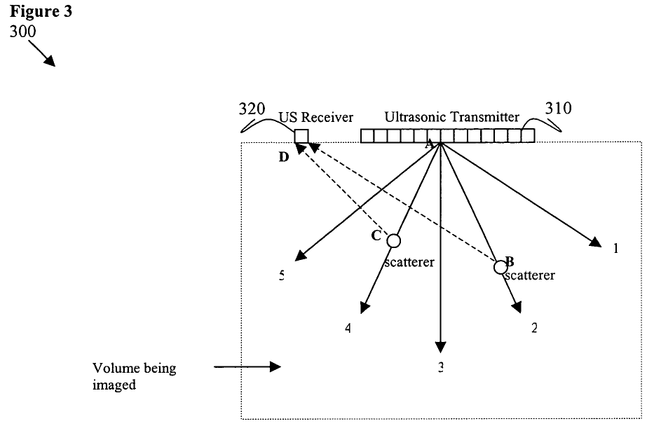

FIG. 3 illustrates an improved ultrasound transducer module 300 for use in an ultrasound imaging system in accordance with an embodiment of the present invention. In an embodiment, the transducer module 300 allows ultrasound transmit beams to be fired almost continuously. The transducer module 300 includes a transmitter array 310 and a receiver 320. The transducer module 300 may be used with the imaging system 100. For example, the transducer 10 of the system 100 may include the transducer module 300.

The transmitter array 310 includes one or more transducers used to transmit ultrasound pulses. The transducers in the transmitter array 310 perform transmit beamforming of an ultrasound transmit pulse. The ultrasound transmit pulse(s) are encoded with different codes before transmission. One or more beamformed ultrasound vectors are transmitted by the transmitter array 310 toward a volume being imaged.

The ultrasound receiver 320 includes a single element transducer or a transducer formed from a transducer array. In an alternative embodiment, a transducer in the transmitter array 310 may be dedicated to receiving rather than transmitting. The receiver 320 receives reflected echo signals produced when ultrasound vectors transmitted by the transmitter array 310 are scattered or reflected by a volume being imaged. The receiver 320 does not perform receive beamforming on a received echo signal. Thus, the receiver 320 receives echo signal levels without directional information. The receiver 320 receives echo signals encoded with the codes used with the ultrasound transmit vectors.

A processing system, such as the imaging mode processor 30 or control processor 50, may determine directional information for received echo signals based on the codes used to encode the ultrasound transmit vectors. The processing system may use timing, direction, and encoding of a received echo signal to determine a range and amplitude of a received echo signal. The processing system may read a code from the received echo signal at the ultrasound receiver 320 corresponding to a code used to encode a transmitted ultrasound beam producing the echo signal upon impact with a structure in an object being imaged. The processing system also receives information from the front-end subsystem 20 and/or transmitter array 310, for example, regarding a direction in which the ultrasound beam was transmitted and timing information regarding when the beam was transmitted. The processing system obtains from the receiver 320 a direction from which the echo signal was received and timing information regarding when the echo signal was received. The processing system may then determine an amplitude or strength and a range or distance traveled for the transmitted beam and received echo signal using encoding, directional, and timing information. A plurality of transmitted beams and received echo signals may be used to construct an image volume.

Encoding and decoding of ultrasound transmit signals and received echo signals may be performed in software and/or in hardware. Software processing allows flexibility in encoding of signals and evaluating received echo signals to determine scatter location. Hardware, such as a digital signal processor (DSP) chip may be used for signal encoding/decoding and additional processing. Received echoes may be cross-correlated with codes used to encode the transmitted beams using software and/or hardware. Amplitudes for different coded signals may be derived. Based on transmit and receive timing, scatter object location may be determined without receive beamforming.

In operation, the transmitter 310 or a front-end 20 processor encodes ultrasound vectors with different codes, such as Barker codes, Golay-type bipolar codes, codes with multiple levels (e.g., codes with more than 1s and 0s), and other codes. The coded vectors are beamformed for transmission. The transmitter 310 fires coded ultrasound vectors along different paths or different directions that together comprise an ultrasound image frame. Each vector firing is a coded pulse sequence with a distinct code. A first code used to encode a first vector in a frame is different from a second code used to encode a second vector in the frame. In an embodiment, codes may be reused in a subsequent frame. Distinct codes are used within a frame to delineate a certain volume without reusing codes. Vectors in a frame are fired sequentially. In an embodiment, no appreciable delay occurs between transmitted vectors. Thus, a transducer duty cycle may be close to unity.

As shown in FIG. 3, five vectors, for example, are transmitted from the transmitter array 310. Point A in FIG. 3 represents a phase center of the transmitter array 310. Vector 1 is fired at an angle 1 with a pulse code 1, for example. Vector 2 is fired at an angle 2 with a pulse code 2, for example. In the example of FIG. 3, a volume being imaged includes two scatterers, or objects reflecting ultrasound vectors, indicated by B and C. Each scatterer produces a reflected ultrasound echo beam. The reflected beam from each scatterer arrives at the receiver 320 at point D. That is, the system transmits an ultrasound signal at one point, such as an element of the transmitter 310, and “listens” for an echo signal at another point, such as the receiver 320. The processing system obtains two data points (a transmission point and a reception point) and triangulates using the data points to determine a location in an object being imaged (i.e., a scatterer). Thus, the system examines a scatter point from slightly different angles (i.e., a large receive aperture) and gathers data without beamforming to form a spatially compound image.

At any given time, reflected signals received by the receiver 320 come from a locus of points in a volume being imaged. The following equation may be used to determine a locus of point locations in the volume being imaged:
[Distance(A to x)+Distance(d to D)]/c+(time from zero that a vector with a received code was transmitted)=constant (1).
In Equation (1), A indicates the phase center of the array 310; x and d correspond to scatterers or reflectors, such as B or C; D indicates the receiver 320; and c is a speed of sound in a medium being imaged. The locus of points indicated by x and d in Equation (1) describe a surface. For example, in a two-dimensional image a locus is an arc.

The imaging system 100 or processing system of the transducer module 300 may determine a time when each transmit vector is fired. The imaging system 100 or processing system may also determine a time when each echo signal is received. The system may determine at which angle each coded vector is fired. Therefore, the system may resolve a position of a scatterer producing an echo signal from a transmit vector. An image of the volume or sector may be formed from scatterer positions in a frame. A strength of a received coded signal provides an echogenicity, or measure of an acoustic shadow, for a scatterer. The echogenicity of the scatterer corresponds to an intensity value in an ultrasound image.

Received echoes are code-matched with the codes used to encode the ultrasound transmit vectors. The echo signals are then decoded. A code used to encode an ultrasound vector that results in an echo signal is used to code-match and decode the echo signal. Amplitude or strength information for an echo signal may be used in conjunction with the code used to code the signal to determine a position of a reflector that generated the echo signal.

In an embodiment, the receiver 320 receives a smaller echo signal than that provided in traditional ultrasound imaging systems and does no receive beamforming. The receiver 320 may also receive a multitude of reflected signals simultaneously. Multiple received signals are distinguished at the receiver 320 using spread spectrum techniques, such as code division multiple access (CDMA) spread spectrum techniques. CDMA employs multiple codes to allow a system to distinguish multiple signals that are transmitted and/or received at approximately the same time.

In an embodiment, codes used to encode transmitted ultrasound signals may be reused after a certain time interval. Due to the finite amount of time between use of the same code, echoes received from a depth lower than a cut-off depth may be excluded to reduce range-ambiguity artifacts. In an embodiment, orthogonal frequency division multiplexing (OFDM) may be used to provide multiple frequencies as channels in parallel to achieve a high symbol or code rate. OFDM spread spectrum technique distributes transmitted data over a plurality of frequencies or carriers that are spaced apart at precise frequencies. Precise frequency spacing or orthogonality allows reception of multiple signals at approximately the same time while minimizing interference or confusion between signals.

In two-dimensional imaging, for example, a locus describes an arc in an imaging space. The system identifies an angle at which a coded ultrasound beam was transmitted. An intersection of two locus arcs may be used to determine a scatter point location. Multiple scatter point locations which produce echo signals may be determined to form an image area. In three-dimensional imaging, for example, an intersection of a spherical surface a ray passing through the spherical surface may be used to determine a collection of scatter points from which an ultrasound image may be generated.

Thus, an imaging system 100 may determine at what location in a reference coordinate system an echo signal was produced and a strength of the echo signal. The display processor 80 or other back-end processor assigns a contrast value (a grayscale value, for example) based on signal strength. Post-processing may occur in the back end of the system 100 to further refine image data. For example, a post-processor may provide compensation for variable gain due to attenuation (fine variance).

Certain embodiments provide a fast implementation of a B-mode sector scan frame rate. Code division multiple access schemes and other signal processing techniques provide advantages in ultrasound image acquisition frame rate. In an embodiment, while the spatial peak temporal average (SPTA) intensity of the ultrasound may increase, an image may be obtained using lower powers due to a high sensitivity achievable using coded pulses. The SPTA, or power emitted from an ultrasound transducer, of a diagnostic ultrasound system is limited by the U.S. Food and Drug Administration for patient health and safety reasons. Continual ultrasound emission using a transducer may increase transducer power and transducer self-heating. However, use of codes with transmitted ultrasound signals and received echoes may decrease power used by an ultrasound transducer to transmit and receive signals. That is, for a given power density, more transmit channels may be utilized with codes.

Certain embodiments may be used with two-dimensional areas, three-dimensional volumes, and/or four-dimensional motion images. For three-dimensional and four-dimensional applications, a two-dimensional transducer array may be used instead of a one-dimensional transducer array used to obtain two-dimensional area images. Continuous transmission and reception at a separate location provide very high frame rates and volume rates. Additionally, once an echo signal is received, processing may occur offline in a separate software program and/or hardware system, such as a general purpose computer. Thus, processing of received echo signals and scatterer location determine may occur separately from transmission and reception of signals. Concurrent offline processing of received echo signals provides a high virtual frame rate.

Certain embodiments may be utilized to provide a high frame rate in a variety of ultrasound imaging systems. Any non-destructive imaging formation technique employing ultrasound (e.g., structural, medical, etc.) may benefit from transmission and reception at separate locations without receive beamforming.

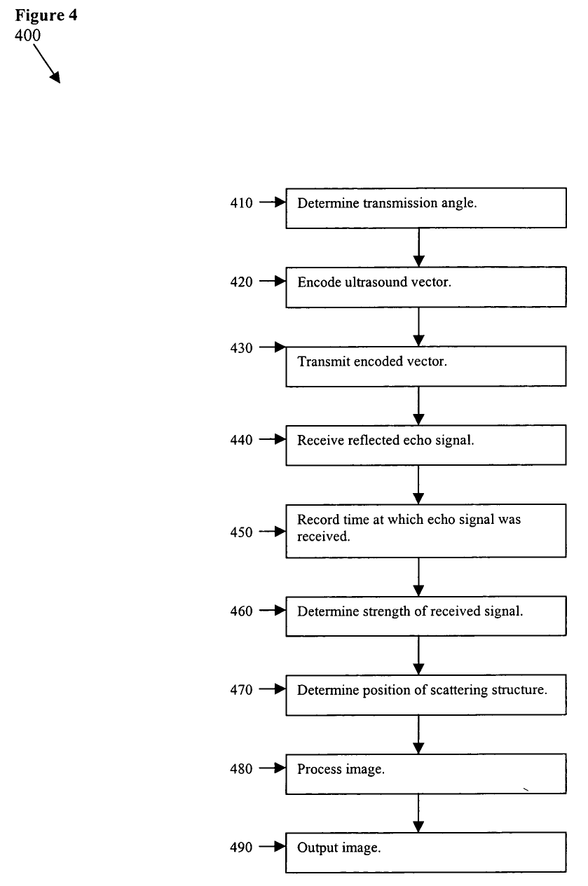

FIG. 4 illustrates a flow diagram for a method 400 for obtaining a very high frame rate in an ultrasound imaging system using separate transmitter and receiver elements in accordance with an embodiment of the present invention. First, at step 410, a transmission angle for an ultrasound beam is determined. For example, an angle may be set manually by an operator or by software operating with the imaging system 100. The angle may be determined according to a type of ultrasound imaging scan or region of interest of an object being scanned.

Then, at step 420, the ultrasound beam vector is encoded with a code. The code may be selected manually by an operator or may be automatically assigned by the system 100. In an embodiment, transmitted ultrasound vectors are assigned distinct codes within a frame. Thus, a vector is encoded with a code that is different from codes used for other vectors comprising a frame. Codes may be reused in a different frame. In an embodiment, a CDMA scheme is used to encode a plurality of vectors.

Next, at step 430, the encoded vector is transmitted by an element of the transmitter array 310. The encoded vector is transmitted at a desired angle toward the object being imaged. An angle at which a vector is transmitted, a time at which the vector is transmitted, and an encoding of the vector may be stored at the system 100. In an embodiment, a plurality of distinctly encoded vectors maybe transmitted at a plurality of angles by elements of the transmitter array 310. Encoded vectors may be fired sequentially, with essentially no delay between vectors. In an embodiment, an OFDM scheme is used to transmit a plurality of encoded vectors. Fired vectors impact structures in the object being imaged. An impact of an ultrasound vector upon a structure may result in a reflected echo signal.

At step 440, one or more reflected echo signals are received at the receiver 320. Then, at step 450, a time at which an echo signal was received at the receiver 320 is recorded. Next, at step 460, the receiver 320 determines a signal level or strength of a received echo signal. Signal level may be measured from an amplitude of the received echo signal. In an embodiment, no beamforming is done to determine directional information from the received echo signal.

Then, at step 470, a position of a structure scattering the coded vector to produce the echo signal(s) is determined. Using the time and angle of transmission, along with the code used to encode the transmitted vector, a distance to a scattering structure may be determined based on the time of reception and strength of the received echo signal. By transmitting a plurality of signals at one location and receiving a plurality of backscattered signals at another location, an ultrasound image of an object may be constructed without receive beamforming. Post-processing may be performed on the image. The image may then be output to a display, stored in a memory, and/or further transmitted. The method 400 results in increased image acquisition frame rates for the imaging system 100.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for improved ultrasound imaging, said method comprising:
encoding an ultrasound signal with a code to produce an encoded ultrasound vector;
transmitting from a first location said encoded ultrasound vector at a desired angle;
receiving at a second location an encoded echo signal produced in response to said encoded ultrasound vector; and
decoding said encoded echo signal using said code used to produce said encoded ultrasound vector.

2. The method of claim 1, further comprising determining a position of a structure producing said encoded echo signal in response to an impact by said encoded ultrasound vector.

3. The method of claim 2, wherein said determining step further comprises determining said position based on a time of transmission of said encoded ultrasound vector, a time of reception of said encoded echo signal, and a strength of said encoded echo signal.

4. The method of claim 3, wherein said determining step further comprises determining said position based on an angle of transmission of said encoded ultrasound vector.

5. The method of claim 3, wherein said time of transmission is determined based on said code.

6. The method of claim 1, further comprising:
transmitting from said first location a plurality of encoded ultrasound vectors at a plurality of angles;
receiving at said second location a plurality of encoded echo signals produced in response to said plurality of encoded ultrasound vectors; and
obtaining an image of an object based on said encoded ultrasound vectors and said encoded echo signals.

7. A method for obtaining ultrasound images at an improved acquisition rate, said method comprising:
encoding a plurality of ultrasound signals for a frame with distinct codes;
sequentially transmitting from a first location said plurality of ultrasound signals at a plurality of angles;
receiving at a second location distinct from said first location a plurality of echo signals formed based said plurality of ultrasound signals; and
decoding said plurality of echo signals using said plurality of distinct codes.

8. The method of claim 7, wherein each echo signal is decoded using a code used to encode an ultrasound signal producing said echo signal.

9. The method of claim 7, wherein said decoding step further comprises decoding said plurality of echo signals offline concurrently with said transmitting step and said receiving step.

10. An improved ultrasound imaging system, said system comprising:
an encoder for encoding an ultrasound signal with a code for transmission to form an encoded ultrasound signal;
a transmitter for transmitting said encoded ultrasound signal; and
a receiver for receiving an encoded echo signal produced based on said encoded ultrasound signal, wherein said receiver decodes said encoded echo signal to produce a decoded echo signal.

11. The system of claim 10, wherein said transmitter comprises a transducer array for transmitting said encoded ultrasound signal.

12. The system of claim 11, wherein said receiver comprises a transducer element of said transducer array, wherein said element is not used for transmitting said encoded ultrasound signal.

13. The system of claim 10, wherein said encoder encodes a plurality of ultrasound signals for transmission with a plurality of codes.

14. The system of claim 13, wherein said codes comprise distinct codes for each ultrasound signal within a frame.

15. The system of claim 10, wherein said transmitter sequentially transmits a plurality of encoded ultrasound signals.

16. The system of claim 15, wherein said transmitter sequentially transmits said plurality of encoded ultrasound signals at a plurality of angles.

17. The system of claim 10, further comprising a processor for determining a position of a scatterer producing said encoded echo signal in response to an impact by said encoded ultrasound signal.

18. The system of claim 17, wherein said processor determines said position of said scatterer based on a time of transmission of said encoded ultrasound signal, a time of reception of said encoded echo signal, and a strength of said encoded echo signal.

19. The system of claim 18, wherein said time of transmission is determined based on said code used to encode said encoded ultrasound signal.

20. The system of claim 17, wherein said processor further determines said position of said scatterer using an angle of transmission of said encoded ultrasound signal.

21. The system of claim 17, wherein said processor determines said position as additional encoded ultrasound signals are transmitted by said transmitter and additional encoded echo signals are received by said receiver.
